(19) United States
(12) Patent Application Publication
(10) Pub. No.: US 2023/0266292 A1
(43) Pub. Date: Aug. 24, 2023

(54) ODOR EVALUATION DEVICE

(71) Applicant: Shimadzu Corporation, Kyoto-shi (JP)

(72) Inventors: Kazuhiro KAWAMURA, Kyoto-shi (JP); Junichi KITA, Kyoto-shi (JP); Motoo KINOSHITA, Kyoto-shi (JP)

(21) Appl. No.: 18/101,652

(22) Filed: Jan. 26, 2023

(30) Foreign Application Priority Data
Jan. 27, 2022 (JP) 2022-011314

(51) Int. Cl. G01N 33/00

(52) U.S. Cl. CPC G01N 33/0032

(57) ABSTRACT

An odor evaluation device according to the present invention includes a gas recovery unit, and a cleaning gas supply unit. The gas recovery unit includes an inlet port configured to introduce a sample gas containing odor components, an outlet port configured to detachably mount a sample bag for recovering the sample gas, a flow path connecting the inlet port and the outlet port, and a switching valve arranged in the flow path to open and close the outlet port. The cleaning gas supply unit is configured to supply a cleaning gas from an upstream side of the switching valve toward the outlet port in a state in which the sample bag is not mounted on the outlet port and the switching valve is opened.

### CROSS-REFERENCE TO RELATED APPLICATIONS

This application claims priority under 35 U.S.C. §119 to Japanese Patent Application No. 2022-011314 filed on Jan. 27, 2022, the entire disclosure of which is incorporated herein by reference in its entirety.

### BACKGROUND OF THE INVENTION

### Field of the Invention

The present invention relates to an odor evaluation device.

### Description of the Related Art

The following description sets forth the inventor’s knowledge of the related art and problems therein and should not be construed as an admission of knowledge in the prior art.

Aromatics to be added to foods, drinks, cosmetics, detergents, and the like are mainly produced by mimicking aromas (natural fragrances) emitted from natural products, such as, e.g., flowers, herbs, and fruits. Many natural fragrances have a composite odor in which a plurality of components is mixed, and a variety of odors are obtained depending on the type and number of components and the proportion of the various components. Further, not all of components constituting natural aromatics do not always contribute to the formation of the odor, but components constituting nature aromatics include components having a very small contribution ratio or components not contributing at all.

As one of methods for identifying whether a component contained in a natural aromatic is a component that contributes to the odor formation or a component that does not contributes to the odor formation, a method using an omission test is known. The omission test is a method in which an omission odor in which any component (or a component group, hereinafter the same) is eliminated from an analysis target odor (composite odor) is prepared, and it is evaluated whether the odor eliminated from the analysis target odor contributes to the odor formation based on the similarity between the emission odor and the analysis target odor.

In the omission test, odor components contained in an analysis target odor are detected as much as possible by using an olfactometry GCMS or the like, and then the quantitative analysis and the qualitative analysis are performed. Then, the detected components are mixed at a ratio corresponding to the quantitative value to prepare a mixed odor. The mixed odor should be the analysis target odor. However, in most cases, it is impossible to detect all of the components contained in the analysis target odor, and therefore, the odor does not actually become the original odor.

Therefore, a perfumer guesses a component lacking in the mixed odor, adjusts the concentration, and add it to the mixed odor to thereby reproduce the analysis target odor. However, in the case of a composite odor, there are components not contributing to the overall odor among the components. Therefore, when the component group reproducing the analysis target odor and the concentration of each component are obtained, a minimum required component group constituting the odor by removing some components from the component group is obtained.

As described above, a conventional omission test requires much expertise and takes much time to identify the component group for reproducing the analysis target odor and the concentration of each component.

On the other hand, for example, Patent Document 1 describes an odor evaluation device capable of efficiently performing processing similar to the above-described conventional omission test in a short time. This odor evaluation device is provided with a gas chromatograph having a separation column for separating an analysis target odor gas, an odor gas recovery unit on which a plurality of sample bags is mounted, a sniffing port for sensorially evaluating an odor gas recovered in each sample bag by sniffing, and an odor sensor for detecting the odor of the odor gas. Then, an index value representing the similarity between the all-component odor gas and the omission odor gas is calculated based on the result of the sensory evaluation or the detection result by the odor sensor.

In the above-described odor evaluation device, the odor gas recovery unit is provided with an inlet port into which components coming out of the separation column are introduced, a plurality of mounting ports on which sample bags are detachably mounted, and a switching valve for guiding the components introduced into the inlet port to any of the plurality of mounting ports.

With such a configuration, it is possible to recover a plurality of types of odor gases (an all-component odor gas and one or more types of omission odor gases) in different sample bags, and therefore, it is possible to sequentially perform the sensory evaluation or the measurement using an odor sensor on the gases recovered in the plurality of sample bags.

### PRIOR ART DOCUMENT

### Patent Document

Patent Document 1: Japanese Unexamined Patent Application Publication No. 2018-036147

### SUMMARY OF THE INVENTION

### Problems to be Solved by the Invention

Some components coming out of the separation column are highly absorptive, and some of them may adhere to the flow path. In an odor evaluation device, normally, an odor gas is recovered in a sample bag, and then a make-up gas for increasing the bulk of the odor gas is supplied to the sample bag. At this time, most of the components adhering to the flow path flow into the sample bag together with the make-up gas. However, in a case where a plurality of mounting ports on which sample bags are mounted is provided, like in the gas recovery unit of the above-described odor evaluation device, the inner wall surface structure of the switching valve is complicated, and therefore, components adhering thereto tend to remain. Further, the components remaining in the switching valve diffuse on the downstream side of the switching valve to adhere to the flow path or adhere to the connecting part between the mounting port and the sample bag. In a case where components are adhering to the mounting port and/or the switching valve, the components are recovered to the sample bag together with the components coming out of the separation column in the subsequent omission test, causing a problem that a sensory evaluation and detection by an odor sensor cannot be accurately performed.

The preferred embodiments of the present invention have been developed in view of the above-mentioned and/or other problems in the related art. The preferred embodiments of the present invention can significantly improve upon existing methods and/or apparatuses.

An object of the present invention is to provide an odor evaluation device capable of preventing unwanted components from being mixed into an odor gas to be recovered in a sample bag.

### Means for Solving the Problems

An odor evaluation device made to solve the above-described problems according to the present invention is provided with:
a gas recovery unit; and
a cleaning gas supply unit,
wherein the gas recovery unit includes:
   an inlet port configured to introduce a sample gas containing odor components;
   an outlet port configured to detachably mount a sample bag for recovering the sample gas;
   a flow path connecting the inlet port and the outlet port; and
   a switching valve arranged in the flow path, the switching valve being configured to open and close the outlet port, and
wherein the cleaning gas supply unit is configured to supply a cleaning gas from an upstream side of the switching vale toward the outlet port in a state in which the sample bag is not mounted on the outlet port and the switching valve is opened.

In the present invention, when the cleaning gas supply unit supplies a cleaning gas from the switching valve toward the outlet port, unnecessary components remaining in the switching valve and unnecessary components adhering to the outlet port are discharged from the outlet port together with the cleaning gas. As the cleaning gas, it is possible to use an inert gas which is odorless or close to odorless and is low in reactivity. For example, it is possible to use, for example, a make-up gas for inflating a sample bag to a predetermined condition used in a conventional odor evaluation device as a cleaning gas.

### Effects of the Invention

In the present invention, it is possible to prevent unnecessary components from being mixed into an odor gas to be recovered in a sample bag.

### BRIEF DESCRIPTION OF THE DRAWINGS

The preferred embodiments of the present invention are shown by way of example, and not limitation, in the accompanying figures.

FIG. 1 is a schematic configuration diagram showing an odor evaluation device according to an example of the present invention.

FIG. 2 is a diagram for describing the order of opening and closing of a plurality of outlet ports provided to a switching valve.

### DETAILED DESCRIPTION OF THE PREFERRED EMBODIMENTS

In the following paragraphs, some preferred embodiments of the invention will be described by way of example and not limitation. It should be understood based on this disclosure that various other modifications can be made by those skilled in the art based on these illustrated embodiments.

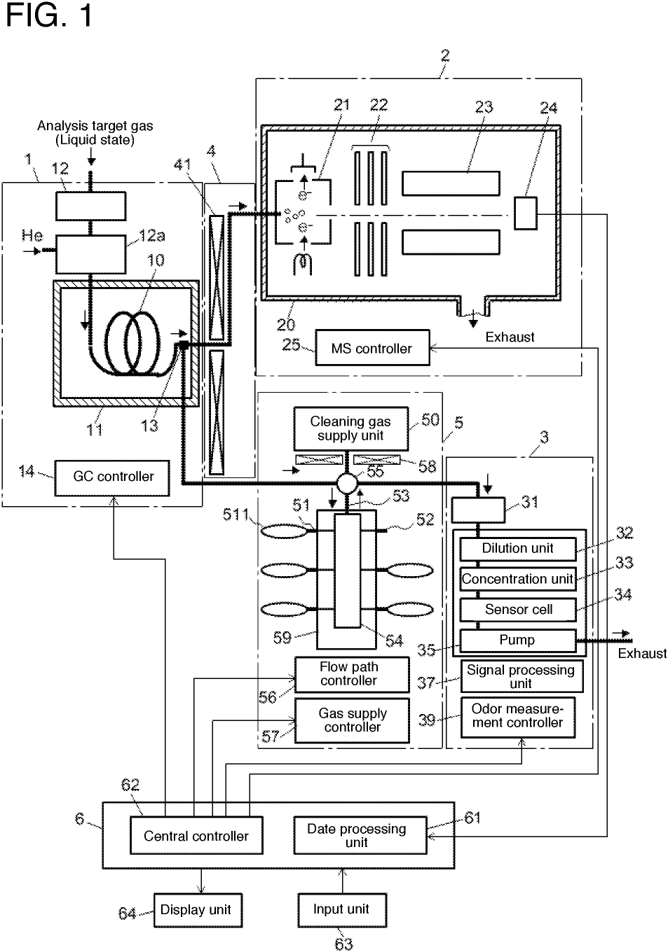

Hereinafter, an odor evaluation device according to one example of the present invention will be described with reference to the attached drawings. FIG. 1 is a schematic configuration diagram showing an odor evaluation device according to this example.

The odor evaluation device of this example is mainly composed of a gas chromatograph unit (GC unit) 1, a mass spectrometry unit (MS unit) 2, an odor measurement unit 3, an interface unit 4, and a gas recovery unit 5.

The GC unit 1 includes a separation column 10, a column oven 11 for accommodating the column 10, a sample introduction unit 12 provided at an inlet of the column 10, a flow path switching unit 13 provided at the outlet of the column 10, and a GC controller 14 for controlling each of these components.

The MS unit 2 includes a vacuum chamber 20, an ion source 21 for ionizing sample component molecules in the introduced sample gas, ion optics 22 for transporting the generated ions, a quadrupole mass filter 23 as a mass separator for separating ions according to the mass number, an ion detector 24 for detecting mass-separated ions, and an MS controller 25 for controlling these components.

The interface unit 4 is provided between the GC unit 1 and the MS unit 2 and includes a heater 41 for maintaining a high temperature in a flow path to facilitate the sample gas flow.

The gas recovery unit 5 is provided between the GC unit 1 and the odor measurement unit 3. The gas recovery unit 5 is provided with: a cleaning gas supply unit 50; a gas recovery unit 59 including a plurality of mounting ports 51 (five mounting ports 51 are shown in FIG. 1) each for mounting a sample bag 511, one discharge port 52, an inlet/outlet port 53, and a first switching valve 54 for switching the flow path between the inlet/outlet port 53 and the mounting ports 51; a second switching valve 55 for switching the flow path between the inlet/outlet port 53 and the odor measurement unit 3, the GC unit 1, and the cleaning gas supply unit 50; a flow path controller 56 for controlling the first switching valve 54 and the second switching valve 55; and a gas supply controller 57 for controlling the cleaning gas supply unit 50.

The above-described one piece of the inlet/outlet port 53 corresponds to the inlet port of the present invention, and the above-described five pieces of the mounting ports 51 each correspond to the outlet port of the present invention. Further, the first switching valve 54 corresponds to the switching valve of the present invention, and the flow path controller 56 corresponds to the open/close controller of the present invention. Although the detailed descriptions will be omitted, the cleaning gas supply unit 50 can also serve as a make-up gas supply unit for supplying a make-up gas to the sample bags 511.

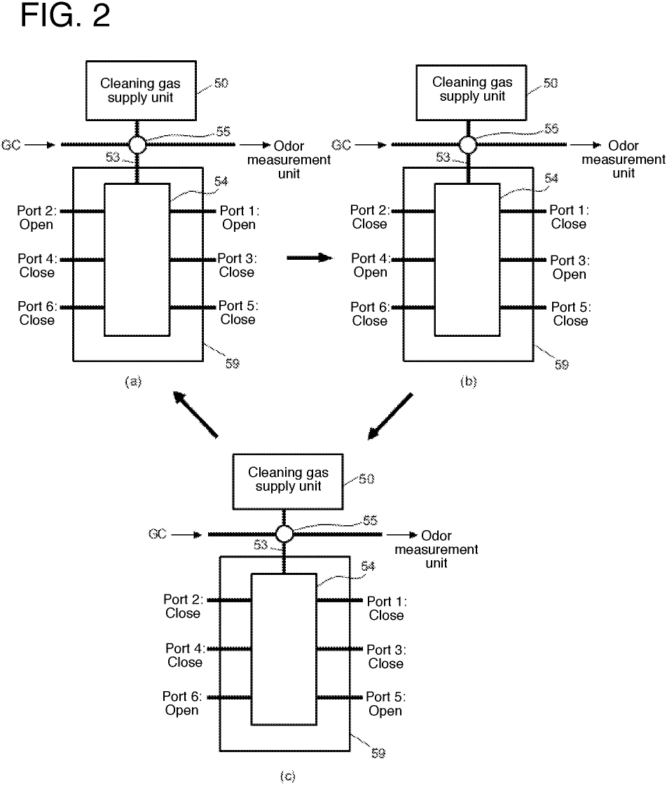

The first switching valve 54 is formed of a multi-port valve having six ports (ports 1 to 6, see FIG. 2). The first port and the discharge port 52 are connected via a pipe, and the second to sixth ports and the five mounting ports 51 are respectively connected via a pipe.

The cleaning gas supply unit 50 is provided with a tank in which a cleaning gas is stored and a pump configured to suck the cleaning gas in the tank and feed it to the inlet/outlet port 53 of the first switching valve 54 (neither of the tank nor the pump is shown). A heater 58 is provided on the outer periphery of the flow path between the pump of the cleaning gas supply unit 50 and the inlet/outlet port 53, and the cleaning gas flowing through the flow path is heated by the heater 58. The heater 58 corresponds to the heating unit of the present invention.

The odor measurement unit 3 is composed of a suction port 31 for suctioning the sample gas, a dilution unit 32 for diluting the suctioned sample gas, a concentration unit 33 for concentrating the suctioned sample gas, a sensor cell 34 provided with a plurality of odor sensors (not shown) for measuring the sample gas containing various odor components, the sensors being different in response characteristic, a pump 35 for drawing the sample gas into the sensor cell 34, a signal processing unit 37 for converting the detection signal by the odor sensor into a digital signal and analyzing the digitized detection signal, and an odor measurement controller 39 for controlling the operation of the entire odor measurement unit 3.

The odor sensor is generally a metal-oxide-semiconductor sensor whose resistivity varies depending on odor components, but may also be any sensor by another detecting mechanism, such as, e.g., a conductive polymer sensor or a sensor in which a gas-adsorbing film is formed on a surface of a crystal resonator or a SAW device.

The signal processing unit 37 and the odor measurement controller 39 are configured mainly by a personal computer 6. In addition to the above, the personal computer 6 includes a data processing unit 61 for analyzing and processing the signal acquired by the ion detector 24 of the MS unit 2 and a central controller 62 for comprehensively controlling the respective controllers 14, 25, 39, and 57. An input unit 63, such as, e.g., a keyboard and a mouse, and a display unit 64 are connected to the personal computer 6.

Further, in this example, when recovering a gas to the sample bag 511, the GC unit 1, the MS unit 2, and the gas recovery unit 5 operate in an integrated manner, and when measuring the odor of the gas recovered in the sample bag 511 with the odor measurement unit 3, the gas recovery unit 5 functions as an autosampler, and the gas recovery unit 5 and the odor measurement unit 3 operate in an integrated manner.

Note that, in this example, the GC unit 1, the MS unit 2, the odor measurement unit 3, and the gas recovery unit 5 (first and second switching valves 54, 55, and the cleaning gas supply unit 50) are controlled by different controllers. However, for example, it may be configured such that the GC unit 1 and the MS unit 2 are controlled by a common controller, and the odor measurement unit 3 and the gas recovery unit 5 are controlled by a common controller.

Next, the basic operation of the odor evaluation device according to this example will be described.

When execution of various operations using the odor evaluation device via the input unit 63 are instructed, the GC controller 14, the MS controller 25, the odor measurement controller 39, and the gas supply controller 57 control, under the control of the central controller 62, the GC unit 1, the MS unit 2, the odor measurement unit 3, and the gas recovery unit 5, respectively.

When an analysis target gas extracted from a gas, a liquid, or a solid sample having an odor is introduced from the sample introduction unit 12 in a gaseous state or a liquid state, the gas is introduced into the column 10 through the sample introduction unit 12. In a case where the analysis target gas is introduced in a liquid state, it is vaporized by the sample introduction unit 12 and then pushed by a carrier gas to be introduced from the sample introduction unit 12 into the column 10. The components contained in the analysis target gas are separated during which it passes through the column 10 and come out of the column 10 in a time-shifted manner. The components coming out of the column 10 are introduced, after passing through the flow path switching unit 13, into the MS unit 2 via the interface unit 4 or into the gas recovery unit 5 via the interface unit 4.

When examining the timing at which the components contained in the analysis target gas come out of the column 10, all the components coming out of the column 10 are introduced into the MS unit 2. Therefore, in this case, the flow path switching unit 13 keeps the GC unit 1 and the MS unit 2 in a communicated state from the time when the introduction of the analysis target gas into the column 10 is started until all the components come out of the column 10. As a result, the components coming out of the column 10 are sequentially introduced into the MS unit 2.

The components introduced into the MS unit 2 are ionized at the ion source 21 under the control of the MS controller 25, and only the ions with a particular mass number selected by the quadrupole mass filter 23 reach the ion detector 24. The quadrupole mass filter 23 repeatedly performs the mass scan within a predetermined mass range, and a detection signal that becomes a source of a mass spectrum is obtained in the ion detector 24 for each scan.

The detection signal acquired by the ion detector 24 is processed by the data processing unit 61, and a mass spectrum in which the horizontal axis represents the mass number and the vertical axis represents the signal strength is repeatedly generated. A total ion chromatogram (TIC) is generated by representing the time by the horizontal axis and representing the signal strength by the vertical axis without considering the mass number. Furthermore, focusing on a certain mass number, a mass chromatogram is generated by representing the time by the horizontal axis and representing the signal strength by the vertical axis.

In order to detect the timing at which the components come out of the column 10, it is sufficient to generate a TIC, but a mass spectrum or a mass chromatogram may be generated as needed. The data of the TIC generated by the data processing unit 61 is stored in the data processing unit 61. Further, the data processing unit 61 extracts peaks from the generated TIC and stores the information (the peak intensity, the peak area, the peak width (time range), and the like) related to the peaks.

Subsequently, the data processing unit 61 automatically sets the timing at which the flow path switching unit 13 causes the GC unit 1 and the gas recovery unit 5 to be communicated with each other and the number of the sample bag 511 at which the components (group) coming out of the column 10 is recovered at the timing so that only the components (group) corresponding to the predetermined time range set in advance is removed from the entire component and recovered in the sample bag 511. The time range in which the components (group) to be removed (omitted) from the entire components (group) come out of the column 10 may be manually set by an operator based on the TIC or the like generated by the data processing unit 61 or may be automatically set by the data processing unit 61. The time range may be set to one or more.

When the time range is set as described above, when recovering the gases to the sample bag 511, the flow path switching unit 13 switches the flow path so that the GC unit 1 and the gas recovery unit 5 are not communicated with each other in the set time range and switches the flow path so that the GC unit 1 and the gas recovery unit 5 are communicated with each other in the other time range. In a case where a plurality of time ranges is set, the gas recovery operation is repeated a plurality of times. In the plurality of gas-recovery operations, the components (group) are recovered in different sample bags 511.

As described above, when a plurality of types of odor measurement gases containing all or a part of components coming out of the column 10 by introducing an analysis target gas into the column 10 is recovered in the sample bag 511, subsequently, the measurement of the measurement gas recovered in the sample bag 511 using an odor sensor by the odor measurement unit 3, or the sensory evaluation using the sniffing port is performed.

When performing the measurement by the odor measurement unit 3, the second switching valve 55 makes the gas recovery unit 5 and the odor measurement unit 3 in a communicated state. Further, the first switching valve 54 opens and closes the second to sixth ports such that the odor measurement gases recovered by the five sample bags 511 are sequentially sent to the odor measurement unit 3. With this, the components of the odor measurement gases in the sample bags are measured.

That is, when the gas to be measured (target gas) is introduced into the sensor cell 34, the components in the target gas come into contact with a plurality of odor sensors, and detection signals different from each other are output in parallel from the respective odor sensors. This detection signal is sampled by an A/D converter, then digitalized, and input to the signal processing unit 37. The signal processing unit 37 acquires one piece of detection data for each odor sensor for one target gas. Therefore, for example, in a case where the sensor cell 34 includes ten (10) odor sensors, ten (10) pieces of detection data will be acquired by measuring one odor measurement gas.

The data processing unit 61 generates an odor vector representing the detection result of each odor measurement gas in the odor space (e.g., ten-dimensional space) based on the detection signal from the odor measurement unit 3. Then, based on the odor vector of each odor measurement gas, the index value representing the similarity between the plurality of odor measurement gases is calculated, and the result is displayed on the display unit 64. At this time, it may be configured such that the type of odor measurement gas by which the index value is calculated and the type of odor measurement gas in which the index value is displayed on the display unit 64 can be selected. Further, it may be configured such that the output value by each odor sensor for each odor measurement gas is displayed on the display unit 64.

Further, by connecting a sniffing port (not shown) to each sample bag 511 removed from the mounting port 51, sensory tests can be performed by a plurality of odor evaluators from a plurality of ports of the gas recovery unit 59. Alternatively, a sensory test can be performed after the gases are collected in the sample bags 511 once. For example, in the case of comparing an odor of a gas (odor measurement gas) containing components (group) recovered in the sample bag 511 with the analysis target gas, a three-point comparison method, which is one of sensory tests, may be used.

When the measurement by the odor measurement unit 3 by the measurement unit 3 or the sensory evaluation of the above-described odor measurement gas has been completed and all of the sample bags 511 have been removed from the mounting ports 51, a GUI indicating an operation button for instructing execution of the cleaning operation is displayed on the display unit 64. Therefore, when the execution of the cleaning operation is desired, the operator operates the input unit 63 to select and operate the operation button. With this, the central controller 62 accepts the execution instruction of the cleaning operation and causes the flow path controller 56 and the gas supply controller 57 to execute the cleaning operation based on the acceptance. That is, in this example, the central controller 62 corresponds to the instruction reception unit of the present invention, and the flow path controller 56 and the gas supply controller 57 correspond to the open/close controller and the cleaning gas supply controller of the present invention, respectively.

Specifically, the flow path controller 56 causes the switching valve 55 to communicate the gas supply unit 50 and the inlet/outlet port 53 of the gas recovery unit 59 with each other. Further, the flow path controller 56 sequentially executes the operation of opening some ports of the first to sixth ports of the switching valve 54 at a predetermined time and closing the remaining ports while switching between the port to be opened and the port to be closed. Further, the gas supply controller 57 operates the pump of the gas supply unit 50 and the heater 58. As a result, the cleaning gas (helium gas) stored in the gas tank is supplied to the switching valve 54.

For example, FIG. 2 shows the temporal change in the opening and closing state of six ports of the switching valve 54 in the cleaning operation. In this example, when the execution of the cleaning operation is initiated, the flow path controller 56 first opens the first and second ports and closes the remaining ports (see (a) of FIG. 2). After maintaining the state shown in (a) of FIG. 2 for a predetermined period of time (e.g., 5 minutes), the flow path controller 56 then maintains the state (see (b) of FIG. 2) in which the third and fourth ports are opened and the remaining ports are closed for a predetermined period of time, and then maintains the state (see (c) of FIG. 2) in which the fifth and sixth ports are opened and the remaining ports are closed for a predetermined period of time.

When the cleaning gas is supplied from the inlet/outlet port 53 into the gas recovery unit 59 in a state as shown in (a) to (c) of FIG. 2, the cleaning gas is discharged to the outside from the mounting port 51 or the discharge port 52 corresponding to the port opened through the inside of the switching valve 54. At this time, the components remaining in the switching valve 54 in the odor measurement gas recovery operation performed immediately before or the measurement operation performed by the odor measurement unit 3, or the components adhering to the mounting port 51 or the discharge port 52 are discharged to the outside together with the cleaning gas.

Further, although the portion of the switching valve 54 related to the port switching is generally complicated in structure, but it is considered that the components adhering to the portion related to the port switching in the switching valve 54 can be easily discharged together with the cleaning gas by repeating the operation of switching the port to be opened as in this example,

Note that in the example shown in FIG. 2, two ports are opened, but one port or three or more ports may be opened. Alternatively, it may be configured such that all of the six ports are opened, and then all of the ports are closed. Furthermore, a series of operations as shown in (a) to (c) shown in FIG. 2 may be repeated a plurality of times.

Further, in this example, the means for instructing the initiation of the cleaning operation is configured by a GUI displayed on the display unit 64. However, in place of it, a mechanical switch provided to the device body may be used. In the above-described example, when one GUI is selected, both the flow path controller 56 and the gas supply controller 57 start executing the cleaning operation, but it may be configured such that the flow path controller 56 and the gas supply controller 57 start individually executing the cleaning operation by another method. Further, it may be configured such that the cleaning operation is automatically executed at an appropriate timing prior to or after the execution of the odor measurement gas recovery operation or after the execution of the odor measurement operation by the odor measurement unit 3. This configuration can be realized by incorporating the step of accepting the execution instruction of the cleaning operation by the central controller 62 into the execution program of the odor measurement gas recovery operation or the execution program of the odor measurement operation.

### ASPECTS

It will be appreciated by those skilled in the art that examples described above are illustrative of the following aspect.

### Item 1

An odor evaluation device as recited in Item 1 is provided with:
a gas recovery unit; and
a cleaning gas supply unit,
wherein the gas recovery unit includes:
   an inlet port configured to introduce a sample gas containing odor components;
   an outlet port configured to detachably mount a sample bag for recovering the sample gas;
   a flow path connecting the inlet port and the outlet port; and
   a switching valve arranged in the flow path, the switching valve being configured to open and close the outlet port, and
wherein the cleaning gas supply unit is configured to supply a cleaning gas from an upstream side of the switching vale toward the outlet port in a state in which the sample bag is not mounted on the outlet port and the switching valve is opened.

According to the odor evaluation device as recited in the above-described Item 1, in a state in which the sample bag is not mounted on the outlet port of the gas recovery unit and the switching value is opened at a predetermined timing after the execution of the gas recovery operation for recovering the sample gas containing all or a part of the odor components contained in the analysis target gas or before executing the recovery operation, the cleaning gas is supplied from the upstream side of the switching value toward the outlet port. Therefore, unnecessary components adhering to the switching valve and/or the outlet port can be discharged from the outlet port together with the cleaning gas. This prevents unnecessary components from entering the gases to be recovered by the sample bag.

### Item 2

The odor evaluation device as recited in Item 2 further includes, in the odor evaluation device as recited in the above-described Item 1:
an instruction reception unit configured to accept an execution instruction of a cleaning operation; and
a cleaning gas supply controller configured to cause the cleaning gas supply unit to execute an operation of supplying the cleaning gas based on that the instruction reception unit accepts the execution instruction.

According to the odor evaluation device as recited in the above-described Item 2, based on the fact that the instruction reception unit accepts the execution instruction of the cleaning operation, it is possible to automatically perform the operation of supplying the cleaning gas from the upstream side of the switching valve toward the outlet port.

### Item 3

According to the odor evaluation device as recited in Item 3, in the odor evaluation device as recited in the above-described Item 1,
the outlet port comprises a plurality of outlet ports, and
the switching valve is configured to individually open and close the plurality of outlet ports.

According to the odor evaluation device as recited in Item 3, it is possible to mount a sample bag on each of the plurality of outlet ports and open or close the plurality of outlet ports in various manners such that all of the plurality of outlet ports are opened and closed collectively, or some outlet ports are opened and closed sequentially.

### Item 4

The odor evaluation device as recited in Item 4 further includes, in the odor evaluation device as recited in Item 3:
an instruction reception unit configured to accept an execution instruction of a cleaning operation; and
an open/close controller configured to cause the switching valve to open a predetermined outlet port out of the plurality of outlet ports for a predetermined period, based on that the instruction reception unit accepts the execution instruction.

According to the odor evaluation device as recited in the above-described Item 4, based on the fact that the instruction reception unit accepts the execution instruction of the cleaning operation, it is possible to open only a predetermined outlet port(s) out of the plurality of outlet ports, such as an outlet port on which the sample bag had been mounted but a sample bag is currently not mounted or a output port on which a sample bag is to be mounted but not currently mounted.

### Item 5

According to the odor evaluation device as recited in Item 5, in the odor evaluation device as recited in any one of the above-described Items 1 to 4,

the cleaning gas is an inert gas.

As the inert gas, a helium gas, a nitrogen gas, and the like, can be exemplified. Some odor evaluation devices capable of amounting a sample bag for odor evaluation include a mechanism to supply a make-up gas, which is an inert gas, in order to inflate the sample bag to a predetermined condition (in order to increase the volume of the gas) after recovering the gas for odor evaluation. In such an odor evaluation device having such a configuration, the make-up gas may be used as a cleaning gas.

### Item 6

The odor evaluation device as recited in Item 6 further includes, in the odor evaluation device as recited in any one of the above-described Items 1 to 5,

a heating unit configured to heat a cleaning gas flowing through a flow path connecting the cleaning gas supply unit and the gas recovery unit.

By heating the cleaning gas, the components adhering to the inside of the gas recovery part and the outlet port can be efficiently discharged.

It should be understood that the terms and expressions used herein are used for explanation and have no intention to be used to construe in a limited manner, do not eliminate any equivalents of features shown and mentioned herein, and allow various modifications falling within the claimed scope of the present invention.

While illustrative embodiments of the invention have been described herein, the present invention is not limited to the various preferred embodiments described herein, but includes any and all embodiments having equivalent elements, modifications, omissions, combinations (e.g., of aspects across various embodiments), adaptations and/or alterations as would be appreciated by those in the art based on the present disclosure. The limitations in the claims are to be interpreted broadly based on the language employed in the claims and not limited to examples described in the present specification or during the prosecution of the application, which examples are to be construed as non-exclusive.

### Description of Symbols

1: GC unit
   10: Column
   14: GC controller
2: MS unit
   25: MS controller
3: Measurement unit
5: Gas recovery unit
   51: Mounting port
      511: Sample bag
52: Discharge port
53: Inlet/outlet port
54: First flow path switching unit
55: Second flow path switching unit
56: Flow path controller
57: Gas supply controller
6: Personal computer
   61: Data processing unit
   62: Central controller
   63: Input unit
   64: Display unit

1. An odor evaluation device comprising:
a gas recovery unit; and
a cleaning gas supply unit,
wherein the gas recovery unit includes:
an inlet port configured to introduce a sample gas containing odor components;
an outlet port configured to detachably mount a sample bag for recovering the sample gas;
a flow path connecting the inlet port and the outlet port; and
a switching valve arranged in the flow path, the switching valve being configured to open and close the outlet port, and
wherein the cleaning gas supply unit is configured to supply a cleaning gas from an upstream side of the switching vale toward the outlet port in a state in which the sample bag is not mounted on the outlet port and the switching valve is opened.

2. The odor evaluation device as recited in claim 1, further comprising:
an instruction reception unit configured to accept an execution instruction of a cleaning operation; and
a cleaning gas supply controller configured to cause the cleaning gas supply unit to execute an operation of supplying the cleaning gas based on that the instruction reception unit accepts the execution instruction.

3. The odor evaluation device as recited in claim 1,
wherein the outlet port comprises a plurality of outlet ports, and
wherein the switching valve is configured to individually open and close the plurality of outlet ports.

4. The odor evaluation device as recited in claim 3, further comprising:
an instruction reception unit configured to accept an execution instruction of a cleaning operation; and
an open/close controller configured to cause the switching valve to open a predetermined outlet port out of the plurality of outlet ports for a predetermined period, based on that the instruction reception unit accepts the execution instruction.

5. The odor evaluation device as recited in claim 1,
wherein the cleaning gas is an inert gas.

6. The odor evaluation device as recited in claim 1, further comprising:
a heating unit configured to heat a cleaning gas flowing through a flow path connecting the cleaning gas supply unit and the gas recovery unit.
